(19) United States
(12) Patent Application Publication
(10) Pub. No.: US 2008/0206186 A1
(43) Pub. Date: Aug. 28, 2008

(54) Pro-Angiogenic Polymer Scaffolds

(75) Inventors: Mark J. Butler, Toronto (CA); Michael Vivian Sefton, Toronto (CA); Gary Alan Skarja, Toronto (CA)

Correspondence Address: BORDEN LADNER GERVAIS LLP;Anne Kinsman, WORLD EXCHANGE PLAZA, 100 QUEEN STREET SUITE 1100, OTTAWA, ON, K1P 1J9, CA

(73) Assignee: RIMON THERAPEUTICS LTD., Toronto, ON (CA)

(21) Appl. No.: 11/912,802

(22) PCT Filed: Apr. 7, 2006
(86) PCT No.: PCT/CA2006/000533

§ 371 Date: Oct. 26, 2007

Related U.S. Application Data
(60) Provisional application No. 60/674,689, filed on Apr. 26, 2005.

(51) Int. Cl. A61K 31/74; A61P 43/00

(52) U.S. Cl. 424/78.31

(57) ABSTRACT

A pro-angiogenic porous polymer scaffold is disclosed. The polymer has at least 20 mol-% monomeric subunits containing acidic functional groups, and has a porosity of at least 40%. The pores in the scaffold are interconnected. A method of making such a scaffold using a novel adaptation to the traditional solvent casting/particulate leaching technique is also disclosed. The scaffold may be used for tissue regeneration.

### FIELD OF THE INVENTION

The present invention relates to a novel porous polymer scaffold, useful for generating a vascularized tissue construct for tissue engineering/regeneration applications.

### BACKGROUND OF THE INVENTION

The emerging fields of tissue engineering and tissue regeneration typically require the intimate interaction of tissue or tissue components and synthetic materials to produce a desired therapeutic effect (e.g. formation of artificial skin to treat extensively burned patients). Synthetic polymers, formed into porous constructs, are often used to encourage tissue ingrowth upon implantation or are seeded with relevant cells prior to implantation to promote new tissue formation. Ideal tissue engineering construct materials must have both appropriate mechanical/physical and biological properties. Appropriate mechanical/physical properties may be attained through the careful selection of polymer chemical composition as well as methods for porous construct formation.

Porous construct formation may be attained in a number of ways. For example, solvent casting/salt leaching is a well-documented technique used to prepare porous, polymeric constructs for tissue engineering applications (Lin, H. R., Kuo, C. J., Yang, C. Y. and Wu, Y. J., “Preparation of macroporous biodegradable PLGA scaffolds for cell attachment with the use of mixed salts as porogen additives”, Journal of Biomedical Materials Research 63(3) 271-279 (2002).; and Murphy, W. L., Dennis, R. G., Kileny, J. L. and Mooney, D. J., “Salt fusion: An approach to improve pore interconnectivity within tissue engineering scaffolds” Tissue Engineering 8(1) 43-52 (2002)). In this technique, a porogen, such as NaCl crystals, is added to a polymer solution and cast into a mold. The solvent is evaporated, resulting in a solid polymer/porogen mixture. Removal of the porogen (e.g. by dissolution in water) results in the formation of a porous polymeric construct.

Porous polymer constructs may be produced in either biodegradable or biostable forms in accordance with the needs of the particular application. Polymers may be rendered degradable through the introduction of readily hydrolysable linkages (e.g. ester, anhydride, amide) to the backbone. Cleavage of the hydrolysable linkages liberates soluble products that, if of the appropriate molecular weight, may be eliminated via normal biological processes. The rate of degradation can be modified by alteration of the polymer chemistry and amount of degradable linkages present in the polymer. In contrast, biostable constructs may be produced by the incorporation of non-degradable linkages (e.g. alkane, ether).

One of the limitations of tissue engineering constructs is that the cells contained within the structure cannot survive unless an oxygen source is within close proximity. Therefore, to prepare functionally useful tissue replacements, new blood vessels must penetrate the scaffold allowing the transport of oxygen and nutrients, preserving viability. New blood vessel ingrowth, also known as vascularization, may be promoted through the local delivery of pro-angiogenic growth factors (e.g. VEGF, FGF). However, these compounds are typically expensive, have short in vivo half-lives and often do not promote the formation of functional blood vessels, at least as individual molecules (Kumar, R., Yoneda, J., Bucana C. D. and Fidler, I. J., “Regulation of distinct steps of angiogenesis by different angiogenic molecules”, International Journal of Oncology, 12(4) 749-757 (1998); and Zisch, A. H., Lutolf, M. P. and Hubbell, J. A., “Biopolymeric delivery matrices for angiogenic growth factors”, Cardiovascular Pathology, 12(6), 295-310 (2003)). Thus, there exists a need for scaffolds which promote vascularization without the addition of pro-angiogenic growth factors.

Pro-angiogenic polymers are known; however, these are not suitable as scaffolds. U.S. Pat. No. 6,641,832 (Nov. 4, 2003 to Sefton et al) describes polyacrylates for use in promoting localized, functional angiogenesis. The polymers were prepared by polymerizing 90 mol-% methyl methacrylate (CH₂=CH(CH₃)COOCH₃) with 10 mol-% methacrylic acid (CH₂=CH(CH₃)COOH) in solution. The resulting polymers were used to make microcapsules (polymeric membranes encapsulating cell(s)) and microspheres (polymeric sphere, typically 10 to 200 microns in diameter). The polymers have pro-angiogenic characteristics but are not suitable as pro-angiogenic scaffolds due to various factors, including their lack of pores, their low acid content (which makes less angiogenic), and they are too brittle.

Acid-containing scaffolds are known (for example Baier Leach J. et al. “Photocrosslinked hyaluronic acid hydrogels: natural, biodegradable tissue engineering scaffolds” Biotechnol. Bioeng. 2003 82:578-89). However, these are not suitable to due their lack of pores.

### SUMMARY OF THE INVENTION

Accordingly, it is an object of the present invention to provide scaffolds, capable of promoting a localized angiogenic response in tissue in the absence of exogenous growth factors. The scaffolds may be degradable or biostable.

Thus, in one aspect, the invention provides a pro-angiogenic porous polymer scaffold. The polymer comprises at least 20 mol-% monomeric subunits containing acidic functional groups, is optionally crosslinked, has a porosity of at least 40%, and has interconnected pores.

In another aspect, the invention provides a method for making a pro-angiogenic porous polymer scaffold, wherein said polymer comprises acidic functional groups grafted to or incorporated into the polymer, said scaffold having a porosity of at least 40% and said pores being interconnected. The method comprises mixing one or more types of monomers and an initiator together in a solvent, wherein at least one of said monomers contains an acidic functional group; pouring the mixture over a fused salt bed having a pore size range of 10 to 800 microns; allowing the mixture to polymerize; and leaching the salt out, to yield the porous scaffold.

Other objects of the present invention will become apparent to those ordinarily skilled in the art upon review of the following description of specific embodiments of the invention.

### BRIEF DESCRIPTION OF THE DRAWINGS

FIG. 1 is an illustration of a network pro-angiogenic polymer.

FIG. 2 is an illustration of a grafted polymer, where the grafts contain acidic functionality making the polymer pro-angiogenic.

FIG. 3 shows a schematic illustrating a salt-bed polymerization method for obtaining porous constructs.

FIG. 4 shows scanning electron micrographs of a poly(MAA-BMA) scaffold (0.10 monomer to salt ratio, 24 h fusion time) cross-sections at two magnifications (40× and 150×).

FIG. 5 shows scanning electron micrographs for poly(MAA-BMA) scaffolds produced using varying salt fusion times: A) 0 h, B) 24 h, C) 48 h and D) 96 h.

FIG. 6 shows the relationship between salt fusion time and the compressive modulus for poly(MAA-BMA) scaffolds (10% monomer to salt ratio).

FIG. 7 shows the relationship between salt fusion time and the yield strength for poly(MAA-BMA) scaffolds (10% monomer to salt ratio).

FIG. 8 shows the effect of monomer to salt ratio on poly(MAA-BMA) scaffold porosity (24 h fusion time).

FIG. 9 shows the relationship between monomer to salt ratio and compressive modulus for poly(MAA-BMA) scaffolds (24 h salt fusion time).

FIG. 10 shows the relationship between monomer to salt ratio and yield strength for poly(MAA-BMA) scaffolds (24 h salt fusion time).

FIG. 11 illustrates the sites of implantation for the test and control scaffold disks.

FIG. 12 shows tissue ingrowth into control and test scaffolds (H+E stained) at 7, 21 and 30 days post-implantation. Poly(MAA-BMA) at 7 days (a), 21 days (c) and 30 days (e). Poly(BMA) at 7 days (b), 21 days (d) and 30 days (f). Scale bars represent 250 μm.

FIG. 13 shows H+E stained scaffold explants at 30 days post-implantation that indicate differences in the inflammatory response for test and control implants. More foreign-body giant cells shown (by arrows) in the poly(BMA) explants (b and d) in comparison to poly(MAA-BMA) (a and c). For figures a and b, scale bar represents 200 μm and figures c and d, scale bar represents 100 μm.

FIG. 14 shows microvessel density counts at 21 and 30 days post-implantation in the pores of test poly(MAA-BMA) and control poly(BMA) scaffold explants. Values represent means ± standard deviations and * represents statistical significance relative to the poly(BMA) control.

FIG. 15 shows fVIII-stained explant samples at 7, 21 and 30 days post-implantation indicating greater vascularisation of the poly(MAA-BMA) scaffolds (a,c and e) in comparison to the control poly(BMA) scaffolds (b,d and f). 7 day samples (a and b), 21 day (c and d) and 30 day (e and f). P denotes areas occupied by polymer scaffold. Scale bars represent 100 μm.

### DETAILED DESCRIPTION

Generally, the present invention provides a new type of porous, polymeric scaffolds containing pro-angiogenic components that can be used for tissue engineering/regeneration applications, a method for making the scaffolds, methods of using the scaffolds, and systems formed from, or incorporating, the scaffolds. Both biostable and biodegradable polymer constructs are contemplated. The scaffold is formed from a pro-angiogenic polymer by incorporating pores.

### The Polymer

The polymer that composes the scaffold is a biocompatible polymer. Biocompatible polymers are defined herein as polymers that induce, when implanted, an appropriate host response given the application. For the purposes herein, they are essentially non-toxic, non-inflammatory, non-immunogenic, and non-carcinogenic.

Furthermore, the polymer encourages vascularization. The term “vascularization” refers to the blood vessel network in and around an implanted scaffold, or the formation of such a blood vessel network.

In order to function as a scaffold, the polymer must be insoluble in aqueous solution at 37° C. (i.e. body temperature).

The polymer is made from polymerizable monomeric subunits or monomers which are polymerized together. The monomers once incorporated into the polymer are referred to herein as mers or monomeric (sub)units. The polymer comprises of the scaffold comprises at least 20 mol-% monomeric units (i.e. mers) contain acidic functional groups. The polymer may contain at least 30, at least 40, at least 45, or at least 50 mol-% of acidic mers. Preferably, the polymer contains at least 45 or at least 50 mol-% of acidic mers. The polymer may comprise 100 mol-% acidic mers, and may be a homopolymer of one type of such acidic mers. However, the polymer will typically contain other biocompatible mers to give the scaffold the desired structural and physical properties, such as solubility, flexibility, strength, etc. These other mers are referred to herein as the backbone mers (though the majority or the entirity of the polymer may consist of acidic mers). Furthermore, the polymer optionally contains crosslinks.

The polymer is preferably a polyacrylate.

The polymer may be biodegradable or biostable.

Examples of suitable copolymer structures are random, block, and graft copolymers.

In the case of a graft copolymer the polymer comprises a backbone and arms grafted onto the backbone. Preferably, the arms contain the at least 20 mol-% monomeric subunits containing acidic functional groups. Methods of making graft copolymers are known in the art. As an example of a graft copolymer, the acidic mers may be grafted to a biocompatible polymer. In this way, a pro-angiogenic effect is conferred to the existing biocompatible polymer. This may be accomplished through the inclusion of grafting sites (e.g. unsaturated carbon bonds, acids, amines, amides, hydroxyls) in the biocompatible polymer.

However, this invention is not meant to include scaffolds which are surface-modified or polymers which are derivativatized post-scaffold formation.

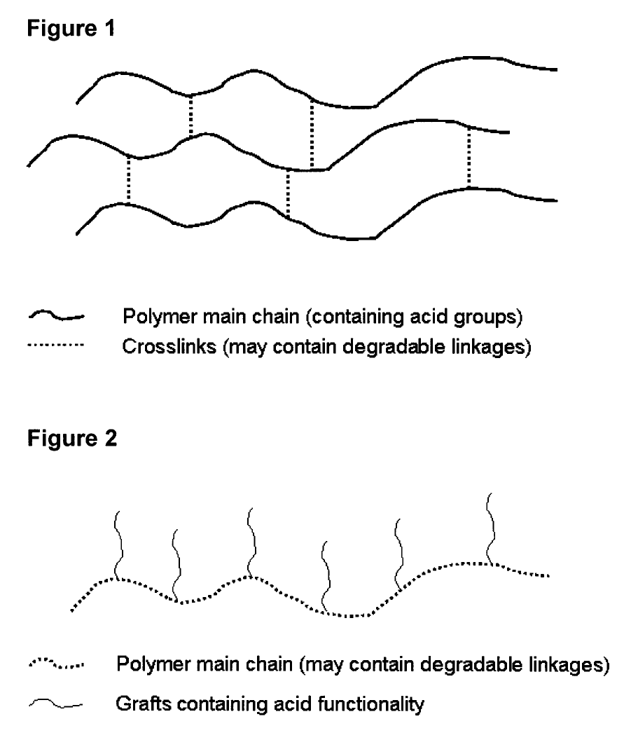

FIG. 1 shows a schematic example of a polymer in accordance with invention with both the acidic and backbone co-monomers used to form the main chain. Degradable cross-links are used to join the various main chains. FIG. 2 shows a schematic representation of a type of graft copolymer in accordance with the invention with the backbone co-monomers joining together to form the main chain and the acidic co-monomers used to make polymers which are grafted onto the main chain.

### Acidic Mers

At least 20 mol-% of the monomeric units (i.e. mers) in the polymer contain acidic functional groups that, upon implantation, bind and stabilize endogenous pro-angiogenic growth factors (such as VEGF and FGF). This provides a sustained, localized angiogenic effect by stabilizing the growth factors (in analogy to extracellular matrix components) and slowly releasing them over a prolonged period of time. Examples of suitable acidic functional groups include any biocompatible acids, such as carboxylic acids (—COOH), sulfonic acids (—SO₃H), and phosphoric acids (—OP(OH₃), and their corresponding salts (i.e. carboxylates (—COO—), sulfonates(—SO₃⁻), and phosphates). Examples of polymerizable groups (i.e. monomers or polymerizable monomeric (sub)units) containing acidic functional groups that may be used to produce the pro-angiogenic polymer of the invention include: acrylates (CH₂CR¹COOR²) (such as methacrylic acid (CH₂C(CH₃)COOH) and acrylic acid (CH₂CHCOOH)), 2-propene-1-sulfonic acid (CH₂C(CH₃)CH₂SO₂OH), 4-vinyl benzoic acid (CH₂—CH—C₆H₄—COOH), crotonic acid (CH₃CHCHCO₂H), itaconic acid (CH₂C(CH₂CO₂H)CO₂H), vinylsulfonic acid (CH₂CHSO₃H), vinyl acetic acid (CH₂CHCHCOOH), citric acid (C(OH)(CO₂H)(CH₂CO₂H)₂, and styrene sulfonic acid (CH₂—CH—C₆H₄—SO₃H), and their salts, such as sodium styrene sulfonate (CH₂—CH—C₆H₄—SO₃Na) and monoacryloxyethyl phosphate. Combinations of the above may also be used. In one aspect, the acidic mers are methacrylic acid. These polymerizable groups may be incorporated directly into the polymer backbone or grafted to the backbone.

### Backbone Mer

In addition to the acidic mer or mers, the polymer may comprise one or more additional non-acidic mers. Any mers may be used so long as the resulting polymer is biocompatible and so long as the starting monomer is polymerizable with the selected starting acidic monomer (i.e. the polymerizable groups (i.e. monomers) containing acidic functional groups). Generally, the mers will be chosen as a function of the desired physicochemical properties (e.g. mechanical, aqueous swelling, etc.), as a function of desired physical properties (such as mechanical strength), and as a function of desired solubility properties, i.e. they may help render the polymer insoluble in aqueous solution at 37° C. Such co-monomers are known in the art.

Examples of backbone co-monomers for forming the polymers of the present invention include acrylates (such as hydroxyethyl methacrylate, methyl methacrylate, butylmethacrylate, hexylmethacrylate, and butylacrylate), phosphazenes, various vinyl co-monomers including vinyl chloride, acrylonitrile, vinyl acetate, ethylene vinyl acetate, vinyl alcohols, vinyl amines, imides, ether ketones, sulphones, siloxanes, urethanes and amides, carbonates, esters and bioresorbables such as anhydrides, orthoesters, caprolactones, amino acids, lactic/glycolic acid co-monomers and hydroxybutyrates. Combinations of the above may also be used.

As a matter of practicality, if the acidic mer is an acrylate, such as methacrylic acid, the backbone co-monomer may be chosen to be an acrylate, such as butyl methacrylate (BMA). The acrylates provide a diverse range of monomers, and are readily available making it possible to tailor material properties to a variety of applications.

### Crosslinkers

The polymer forming the scaffold is optionally crosslinked. Crosslinking is used to render the polymer insoluble in aqueous solution at 37° C. The crosslinks may be biodegradable or biostable. The crosslinking agent is generally incorporated into the polymer comprising the scaffold during polymerization, in an amount of about 0.001 to about 5 mol-% based on the total number of mols of monomers comprising the polymer, preferably about 0.01 to about 1 mol-%. The amount of crosslinker chosen will depend on the desired physicochemical properties of the resultant scaffold including, in the case of the degradable linkers, the rate of degradation desired.

Biostable crosslinking agents: Biostable crosslinking agents are known in the art. Examples of biostable crosslinking agents are biocompatible divinyl benzenes and bifunctional acrylates, such as (poly)ethylene glycol dimethacrylates, e.g. ethylene glycol dimethacrylate (EGDMA). An advantage of polyethylene glycol dimethacrylates is that the length of the polyether chain can be modified to suit the application.

Degradable linkages: In many cases it may be desirable to have the constructs degrade in vivo over time. Degradable constructs can be produced through the incorporation of crosslinkers that contain hydrolysable linkages (i.e. ester, amide, anhydride). Cleavage of these crosslinks by simple chemical or enzyme-mediated hydrolysis breaks down the polymer network, liberating soluble polymer chains, which eventually leads to the elimination of the solid construct. The rate of polymer degradation may be modified through the selection of monomer chemistry, crosslinker chemistry and crosslink density. Crosslinker molecules containing internal hydrolysable linkages (e.g. ester, amide, anhydride) and polymerizable functional groups, yielding an overall functionality greater than 2, introduce degradable branch points in the formation of insoluble, network polymers. These crosslinkers are obtained by covalently attaching polymerizable functional groups to the ends of molecules containing degradable linkages. The attached polymerizable functional groups may include: methacrylate, acrylate, isocyanate, carboxylic acid, acid chloride, vinyl, amine, and hydroxyl. An example of commonly used degradable linkers is methacrylated polyesters, such as polycaprolactone, which liberates non-toxic degradation products.

### The Scaffold

The scaffold must have a porosity of at least 40%. For many applications it is preferred to have a porosity of at least 70%, preferably at least 80%. A porosity of at least 90% may also be desirable. The porosity (pₒ) is calculated as: pₒ=1−(d/dₚ), were dₚ is the density of the non-porous scaffold, and d is the density of the porous scaffold. The density of the scaffolds (d) is calculated as d=m/v (where m is the mass and v the volume); alternatively, literature values for the density of non-porous scaffolds may be used.

The pore diameter (primary pores) will generally be between 10 to 800 microns, with the average pore diameter being between 200 to 350 microns; though for certain applications a range of 25 to 250 microns may be preferred.

The pores of the scaffold are interconnected. The diameter of the interconnections is significantly smaller than the pore diameter, typically less than about 100 microns. The pores must be sufficiently interconnected to permit vascularization.

In one particular embodiment, the invention provides a pro-angiogenic porous polymer scaffold, said polymer being a polyacrylate comprising at least 20 mol-% monomeric subunits containing acidic functional groups, said polymer being optionally crosslinked, having a porosity of at least 40%, and having interconnected pores. The monomeric subunits containing acidic functional groups may be methacrylic acid. The mol-% of monomeric subunits containing acidic functional groups may be at least 45 mol-%. The backbone mers may be one or more types of methacrylates, such as butylmethacrylate.

### Methods of Making the Scaffold

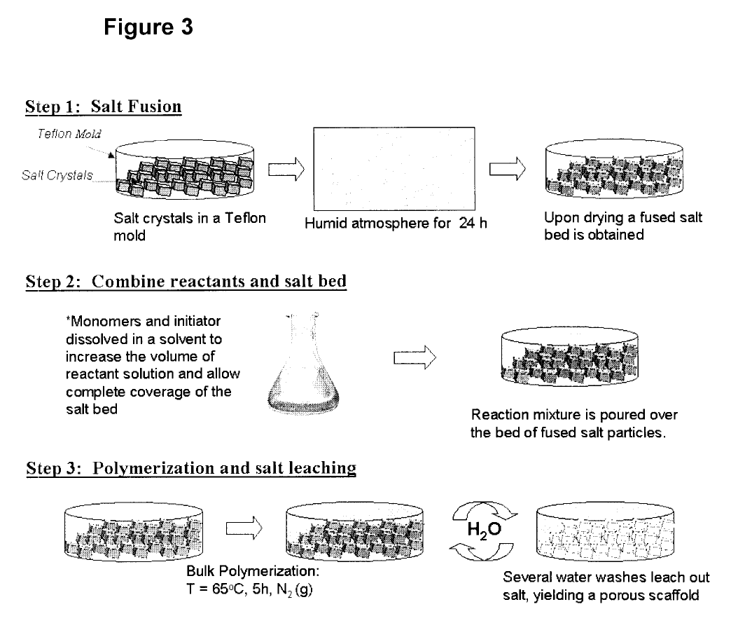

A novel method for making scaffolds is disclosed, using a modified porogen technique, as described in more detail in Example 1. Generally, the monomers, optionally the crosslinker, and the initiator are dissolved in a solvent, poured into a bed of fused particles (such as a salt) and polymerized. As the polymerization and optionally crosslinking reaction proceeds, the polymer precipitates out of solution. The solvent is removed. Removal of the included fused particles (such as salt crystals) results in a highly porous polymer construct. The method is illustrated in FIG. 3.

More specifically, the particles are fused by exposing them to a humid environment for a predetermined length of time. As is discussed in Example 3, longer fusion times result in progressively less organized pore structures and increasing frequency of holes in the primary pore walls of the scaffold.

Examples of suitable particles include sugars, such as glucose, and organic and inorganic salts, such as NaCl. NaCl is preferred.

Particles having a diameter corresponding to the desired diameter of the pores in the scaffold are suitable. For instance, the particles may have a particle size of about 10 to 800 microns, with the average diameter being between 200 to 350 microns; though for certain applications a range of 25 to 250 microns may be preferred. The particles can be sorted by size prior to fusion depending on the desired average pore size and size ranges.

The monomers, initiator, and optionally crosslinking agent are combined in a suitable solvent, such as methylene chloride, ethyl acetate, chloroform, acetone, benzene, 2-butanone, carbon tetrachloride, n-heptane, n-hexane, and n-pentane. For polyacrylates, chloroform is often suitable. The mixture is poured over the fused particle bed and is allowed to polymerize under conditions suitable for the particular polymer chosen.

The monomer to particle ratio is selected to achieve the desired porosity. For instance, it may range from 7 to 16% wt:wt expressed as a percentage.

Once the polymerization is complete the solvent is removed, such as by evaporation (such as by air drying).

The scaffold is then subjected to one or more washes with a solvent in which the particles are soluble, but the scaffold is not, such as water.

Thus, in one aspect, the invention provides a method for making a pro-angiogenic porous polymer scaffold, wherein said polymer comprises acidic functional groups grafted to or incorporated into the polymer, said scaffold having a porosity of at least 40% and said pores being interconnected, said method comprising: mixing one or more types of monomers and an initiator together in a solvent, wherein at least one of said monomers contains an acidic functional group; pouring the mixture over a fused salt bed having a pore size range of 10 to 800 microns; allowing the mixture to polymerize; and leaching the salt out, to yield the porous scaffold.

Other methods for making porous scaffolds are known in the art (Sachlos E. Czernuszka J. T., “Making Tissue Engineering Scaffold Work. Review on the Application of Solid Freeform Fabrication Technology to the Production of Tissue Engineering Scaffolds” European Cells and Materials Vol. 5 2003, 29-40) and could be used to make scaffolds of the present invention using the pro-angiogenic polymers described herein. These include gas foaming, fibre meshes/fibre bonding, phase separation, melt moulding, emulsion freeze drying, solution casting, freeze drying, and solid freeform fabrication.

The method of making the scaffold and the monomeric units chosen to be included in the scaffold can vary and will depend on the particular application. These and other methods may be used, so long as the scaffold produced is porous and the pores are interconnected.

### Uses of the Scaffold

There are different approaches to implanting the scaffolds known in the art. These include implantation of the scaffolds alone (known as guided tissue regeneration); seeding the scaffolds with cells in vitro and then implanting them immediately; or seeding the scaffolds with cells in vitro allowing the cells to grow, and then implanting the scaffolds. The target tissues for use with these scaffolds are principally vascularized tissues, such as the skin, the blood, the organs . . . etc. Tissue with little vascularization, such as cartilage, is not preferred.

The scaffold may also be used as a bioreactor, by implanting the scaffold with cells and allowing the cells to produce a given protein; examples of proteins include growth factors. The scaffold has the ability to provide a unique environment for the maintenance of such cells.

The scaffold could also be used to generate artificial organs by placing several cell types into the scaffold and providing organizational cues (i.e. mechanical and/or biochemical stimuli) to promote complex 3-D tissue formation.

### EXAMPLES

### Example 1

### Scaffold Fabrication

A novel adaptation to the traditional solvent casting/particulate leaching technique was used to prepare the porous scaffolds. The monomers were dissolved in solvent and polymerized in situ on a bed of fused salt (NaCl) particles. Subsequent to polymerization, the reaction solvent was evaporated off leaving a polymer-salt composite. Sequential washes in various solutions removed the salt, yielding a porous polymer scaffold.

Salt Fusion: A salt fusion technique was used to generate pore interconnectivity in the fabricated scaffolds (FIG. 3). Pore interconnectivity is essential to allow tissue ingrowth and vacularization upon implantation. The fusion technique involves exposing salt particles to a humid environment prior to scaffold formation. When exposed to the humid environment, adjacent salt crystals fuse in a process called “caking”. The surfaces of contacting salt particles coalesce, forming bridges between particles thereby increasing scaffold pore interconnectivity upon salt dissolution.

Unsieved NaCl (20 g) was added to a PTFE mold and agitated until level. The mold was then placed in a large beaker containing distilled water (1 cm depth). The top of the beaker was sealed with Parafilm® and placed in an oven (37° C.) to create a humid environment. After the desired fusion time (24 to 96 h), the mold containing the fused salt particles was removed from the beaker and dried for 24 h in an oven (37° C.). The degree of salt particle fusion was varied by altering the fusion time.

In Situ Polymerization: The monomers and initiator, namely 45 mol % methacrylic acid, 54 mol % comonomer (meth)acrylate, 1 mol % ethylene glycol dimethacrylate (EGDMA) (the biostable crosslinker), and benzoyl peroxide (an initiator) were dissolved in chloroform. Comonomer (meth)acrylates employed were methylmethacrylate (MMA), butylmethacrylate (BMA), hexylmethacrylate (HMA) and butylacrylate (BA). Chloroform was used as a solvent (at 2:1 chloroform to total monomer volume ratio) to increase the volume of reactant solution to allow complete coverage of the salt bed. The reaction mixture was poured over the bed of fused salt particles. The polymerization reaction proceeded for 5 h at 67° C. under nitrogen gas (FIG. 3). A reflux condenser was attached to the reaction vessel to limit evaporation of the solvent during polymerization. Upon completion of the reaction, the polymer-salt composite was air dried overnight to remove chloroform. A poly(butylmethacrylate) control scaffold was synthesized as above to directly assess the effect of methacrylic acid incorporation on the in vivo response to the scaffolds.

Salt Removal and Scaffold Purification: The salt-containing scaffolds were subjected to a series of water washes to remove the embedded porogen. Scaffolds were placed in deionized water for 5 days, replacing the water at least 3 times per day for a total of 15 washes. Upon salt removal, the scaffolds were dried under vacuum for 24 h. Residual monomers and solvent were removed through a series of acid, base and solvent washes. The scaffold was placed sequentially in the following solutions for 3 h each at room temperature:

| | |
|---|---|
| 1. | 0.1 M HCl |
| 2. | Water |
| 3. | Acetone |
| 4. | Acetone |
| 5. | Acetone |
| 6. | Water |
| 7. | 0.1 M NaOH |
| 8. | 0.5 M HCl |
| 9. | Water |
| 10. | DMF |
| 11. | Water |
| 12. | 0.1 M NaOH |
| 13. | Water |
| 14. | 0.5 M HCl |
| 15. | Water |
| 16. | Water |

The scaffolds were cut into disks (6 mm diameter×2 mm thick) and washed with 95% ethanol to remove endotoxin (lipopolysaccharide fragments of gram-negative bacterial cell walls, which are found as contaminants almost everywhere) (EU). Scaffold pieces (1-2 g) were placed in a 50 mL polystyrene tube and 40 mL of ethanol was added. The tubes were sonicated for 20 min., the ethanol was removed and a fresh 40 mL of ethanol was added to the tube. This washing procedure was repeated 10 times. Following the ethanol washes, the scaffolds were washed with endotoxin-free water to remove residual ethanol. The scaffolds were then dried under vacuum and stored in a desiccator. Endotoxin testing (LAL Pyrochrome Kit, Cape Cod, USA) was performed to ensure the scaffolds contained less than 0.25 EU/mL. Any scaffolds that contained >0.25 EU/mL were rewashed as above until the endotoxin level was below the cut-off value.

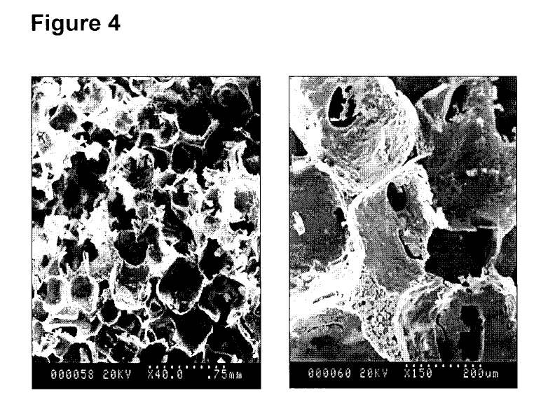

Scaffold Characteristics: The scaffolds were visualized using scanning electron microscopy (SEM) to assess the pore size range and pore interconnectivity. Specimens were frozen in liquid nitrogen for 5 min and cut with a razor blade. Cross-sections of the scaffolds were sputter coated with gold and visualized on a Hitachi S800 scanning electron microscope. FIG. 4 shows scanning electron micrographs of a poly(BMA-MAA) scaffold made with 24 h salt fusion and a 10% weight ratio of monomer to salt. Pore interconnectivity can be seen at higher magnification. Diameters of the primary pores range from approximately 100-600 μm, with the majority falling within the 200-350 μm range. The interconnecting pores resulting from salt fusion were significantly smaller in size (<100 μm).

### Example 2

### Effect of Comonomer Chemistry on Scaffold Properties

MAA-containing scaffold copolymer formulation was examined using four different acrylate comonomers, methylmethacrylate (MMA), butylmethacrylate (BMA), hexylmethacrylate (HMA) and butylacrylate (BA). The mechanical stability of the various copolymer scaffolds was assessed by visual observation during the salt leaching phase of the fabrication process and/or quantitatively evaluated by compression testing. All scaffolds were produced using the following monomer feed ratios: 50 mol % MAA, 49 mol % comonomer and 1 mol % crosslinker (EGDMA).

Qualitative Visual Assessment: Porous scaffolds fabricated with MMA as the comonomer were brittle and crumbled easily with handling during the salt leaching phase. Poly(MAA-MMA) scaffolds fabricated with a monomer to salt ratio of 12.5% or lower disintegrated into small fragments. Poly(MAA-BMA) scaffolds were found to be much less brittle than the poly(MAA-MMA) scaffolds. Mechanically stable (qualitatively assessed) scaffolds were produced down to a monomer-salt ratio of 10%. In comparison, MAA-containing scaffolds produced by copolymerization with hexylmethacrylate and butyl acrylate were much softer and less brittle than either the BMA or MMA versions, as expected. These differences were examined in more detail by compression testing.

Compression Testing: Compressive mechanical properties were measured in a phosphate-buffered saline (PBS) solution at 37° C. on a Mach-1 ™Micromechanical System equipped with a 0.01 kN load cell according to ASTM F541-99a standard specifications for testing acrylic bone cement. Four cylindrical samples (6 mm diameter, 12 mm thick) for each scaffold formulation were preconditioned in PBS at 37° C. for 24 h prior to testing. The specimens were compressed at a rate of 1.0 mm/min up to a strain level of approximately 0.7 mm/mm. Young's modulus (E) was calculated from the stress-strain curve as the slope of the initial linear portion of the curve, neglecting any toe region due to the initial settling of the specimen. The compressive strength at yield (σ_{y}) was defined as the intersection of the stress-strain curve with the modulus slope at an offset of 1.0% strain. A Student's t-test was performed in comparing means from two independent sample groups. A significance level of p<0.05 was used in all the statistical tests performed.

Table I shows the effect of comonomer type on scaffold compressive mechanical properties. Poly(MAA-MMA) scaffolds were not tested since they were too brittle and friable to easily prepare test specimens. Both poly(MMA) and poly(MAA) have glass transitions over 100° C., making the copolymer composed of these monomers rigid. This rigidity combined with the high porosity necessary for a tissue engineering scaffold likely led to the brittle quality of this formulation. All other specimens were produced using a salt fusion time of 24 h and a monomer to salt ratio of 10%. Scaffold stiffness, as indicated by Young's modulus (E), decreases dramatically with comonomer type from BMA to HMA to BA. In addition, compressive strength at yield was only measurable for the BMA-containing copolymer scaffold. HMA has a longer pendant group than BMA which serves to limit chain packing and increase the free volume of the polymer, effectively lowering the glass transition temperature (T_{g}). This results in a weaker, softer copolymer as shown in Table I. BA has a similar chemical structure to BMA, only lacking a methyl substituent group. The absence of this methyl substituent in BA permits greater chain mobility, reducing the T_{g} of the copolymer. This results in a weaker, softer copolymer than both the BMA and HMA-containing ones. This data shows that modifying the comonomer chemistry is a relatively simple method for generating MAA-containing scaffolds with a broad range of physical properties that may be tailored to suit a variety of applications.

TABLE I
Effect of comonomer chemistry on compressive
properties for MAA-containing scaffolds

| | Monomer: | Fusion | | |
|---|---|---|---|---|
| Comonomer | Salt (%) | Time (h) | E (MPa) | σ_{y} (MPa) |
| BMA | 10 | 24 | 1.9 ± 0.3 | 0.15 ± 0.03 |
| HMA | 10 | 24 | 0.7 ± 0.1 | ND |
| BA | 10 | 24 | 0.04 ± 0.01 | ND |

### Example 3

### Modifying Scaffold Porosity and Pore Structure

Copolymer scaffold pore structure and porosity were systematically modified by altering the salt fusion time and monomer to salt ratio (wt/wt, expressed as a percentage) in the reaction mold.

Incubation of NaCl crystals in a humidified environment resulted in fusion of the crystals, creating a highly interconnected salt matrix. Salt fusion times were varied from 0 to 96 h and the resulting scaffolds were visualized by SEM to assess pore morphology. In addition, the effect of salt fusion time on scaffold mechanical properties was determined by compressive testing (done as described in Example 2). All scaffolds tested were poly(MAA-BMA) with a monomer to salt ratio of 10%.

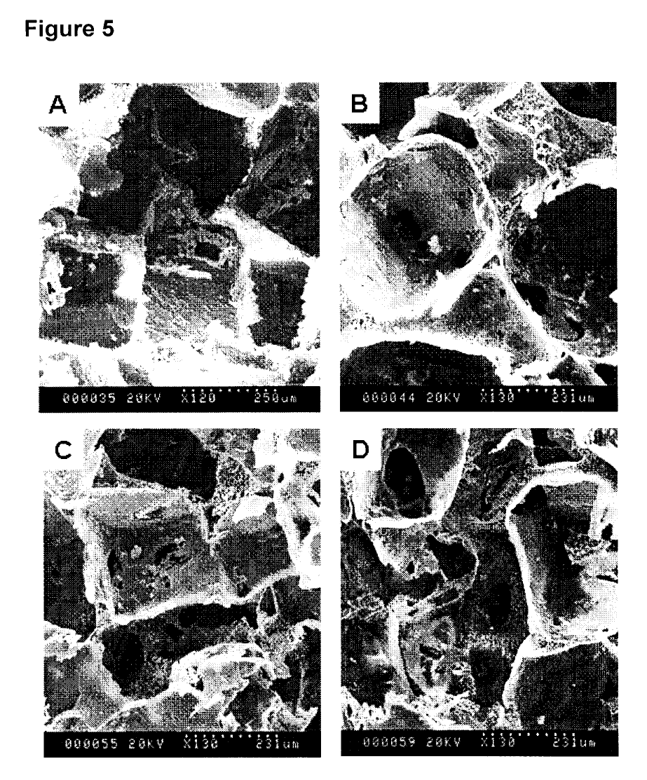

FIG. 5 shows the pore structure of scaffold cross-sections as a function of salt fusion time. The unfused salt scaffold (A) has a well-defined pore structure that appears to be poorly interconnected. In contrast, for the salt fused scaffolds a highly porous and interconnected pore structure is evident. For the 24 h fusion scaffold (B), clearly defined primary pores are seen with holes in the pore walls. Longer salt fusion times (48 h (C) and 96 h (D)) resulted in progressively less organized pore structures and increasing frequency of holes in the primary pore walls. In addition, the holes in the primary pore walls increased in size with salt fusion time. Finally, the pore walls are appreciably thicker in the 24 h salt fusion scaffold, likely a result of larger interstitial space between less fused salt particles that was filled with the copolymer.

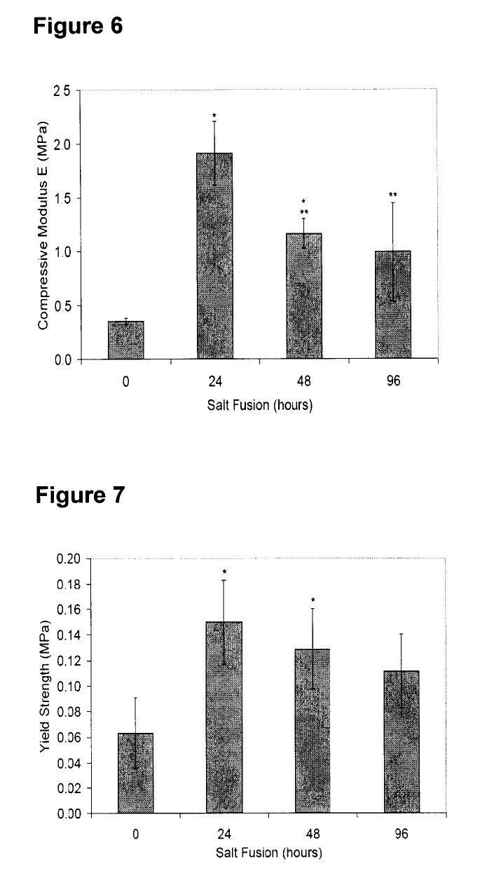

Salt fusion had a pronounced effect on the mechanical properties of the scaffolds. As seen in FIG. 6, scaffolds fabricated with 24 or 48 h salt fusion time were found to have a significantly higher compressive modulus (E) compared with the unfused scaffold. Scaffolds produced with 48 and 96 h salt fusion times were found to have significantly lower moduli compared to the 24 h scaffold. The dependence of yield strength (σ_{y}) on salt fusion time followed a similar trend (FIG. 7). The 24 h salt fusion time scaffold produced a significantly higher yield strength than the unfused scaffold but increasing fusion time resulted in reduced yield strengths. The inter-particle space is larger upon short salt fusion time (i.e. 24 h) due to a small amount of particle erosion that results in a “rounding-off” of the salt particles. The increased inter-particle space is filled during polymerization leading to thicker pore walls and stronger scaffolds. However, as the salt fusion time is increased to 48 and 96 h, the salt particles become increasingly connected; reducing the inter-particle space leads to thinner pore walls and a more disorganized pore structure (seen in FIG. 5). These factors combine to produce the decreasing modulus and yield strength values at the longer salt fusion times seen.

Scaffold porosity was modified by varying the monomer to salt ratio (wt/wt) used in the reaction mold. For this study, poly(MAA-BMA) scaffolds were produced using a salt fusion time of 24 h and the monomer to salt ratio was varied from 7.5 to 15%. The density and porosity of the scaffolds were determined in triplicate by measuring their dimensions and masses. The density of the scaffolds (d) was calculated as follows: d=m/v (where m is the mass and v the volume). The porosity (pₒ) was calculated as: pₒ=1−(d/dₚ), were dₚ is the density of the non-porous polymer (dₚ=1.1 g/cm³ based on literature values).

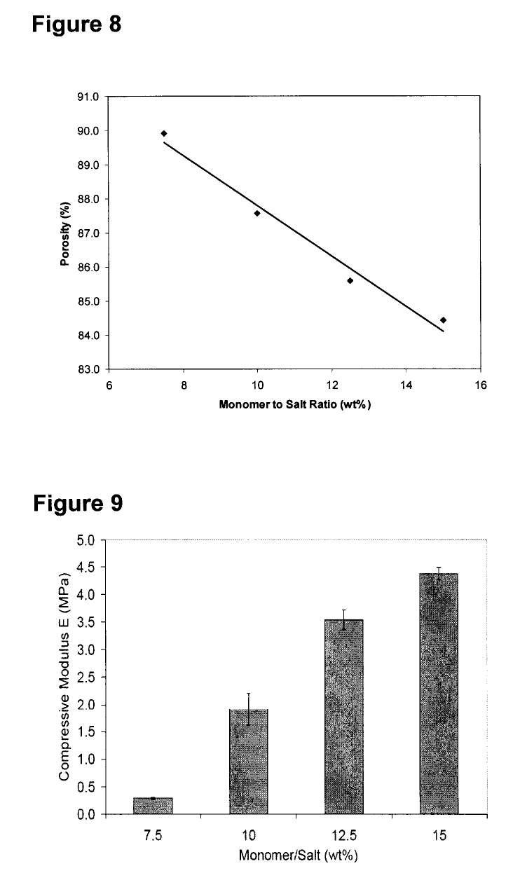

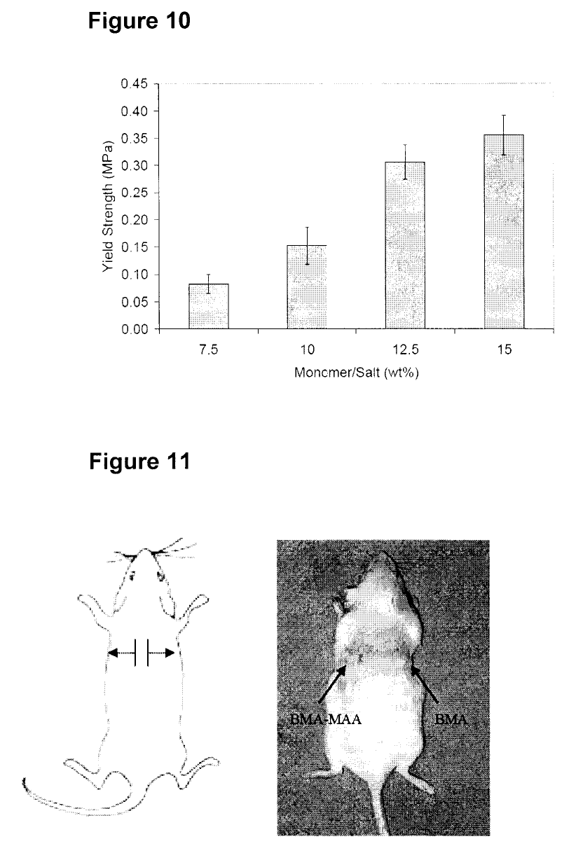

The porosities of the poly(MAA-BMA) scaffolds produced as a function of monomer to salt ratio are shown in FIG. 8. Increasing monomer to salt ratio resulted in decreasing scaffold porosity, as expected. Compressive testing showed that both modulus and yield strength increased with increasing monomer to salt ratio (FIGS. 9 and 10). As expected, increasing scaffold porosity (with decreasing monomer to salt ratio) resulted in decreasing mechanical properties as a result of thicker or more numerous pore walls.

### Example 4

### Scaffold Cytotoxicity

Scaffold cytotoxicity was evaluated prior to implantation studies to assess the effectiveness of the washing method used to remove residual monomers and solvent post-polymerization. An alamarBlue™ cell viability assay (Biosource, USA) was conducted on cells after direct contact with poly(MAA-BMA) scaffolds and contact with a scaffold extract. The alamarBlue™ assay incorporates an oxidation-reduction indicator that changes in color in response to the chemical reduction of the growth medium resulting from metabolic activity. The color change of the cell culture medium is measured spectrophotometrically at two wavelengths.

Scaffold Extract Test: THP-1 monocytes cultured in RPMI medium supplemented with 10% fetal bovine serum were seeded into wells in a tissue culture polystyrene (TCPS) 96-well plate at 3 cell densities (100,000, 150,000 and 250,000 cells/well) and evaluated in triplicate. The cells were differentiated overnight into macrophage-like cells with the addition of phorbol myristate acetate (PMA). The next day the cells were rinsed twice with 150 μL media per well to remove the PMA. Media (150 μL/well), previously incubated with poly(MAA-BMA) scaffold for 48 h (40 mg scaffold/10 mL medium), was then added to each test well while a fresh 150 μL of medium was added to each control well. The cells were incubated for 24 h, then 150 μL of fresh medium and 16.65 μL of alamarBlue™ solution was added to each well. The cells were incubated for a further 4 h, then 100 μL of solution was transferred from each well to a new plate and the solution absorbance was read at 570 and 600 nm to quantify viability. Cell viability by alamarBlue™ assay, when exposed to the poly(MAA-BMA) extracts, was determined to be >100% i 5% compared to cells cultured with fresh media.

Direct Contact Test: THP-1 monocytes were differentiated into macrophage-like cells and seeded in a TCPS plate, as for the extract test. Medium (150 μL/well), containing crushed scaffold (1 mg scaffold/mL medium), was then added to each test well containing activated cells while 150 μL of fresh medium was added to each control well. The cells were incubated for 24 h, then 150 μL of fresh medium and 16.65 μL alamarBlue™ was added to each well and incubated for 4 h. The absorbance of each well was measured directly. Cells cultured directly with the crushed poly(MAA-BMA) scaffolds exhibited a high level of viability (91±7%) compared to cells cultured in fresh media. This result, in conjunction with the scaffold extract result, suggests that the scaffold washing procedure was effective in removing residual monomers and solvent post-polymerization. The slight decrease in viability for cells in direct contact with the scaffold pieces may be attributed to a difference in adherence to the pieces compared to TCPS or a mild inhibitory (non-toxic) effect on cell metabolism by the scaffold fragments.

### Example 5

### In Vivo Evaluation of Scaffolds

The angiogenic potential of the scaffolds was evaluated in a murine subcutaneous implant model. The test scaffolds were all poly(MAA-BMA) produced using a monomer to salt ratio of 10% and 24 h salt fusion time because these conditions produced a well interconnected, highly porous scaffold that was easily handled. Since MAA is the pro-angiogenic component of the copolymer, homopolymer poly(BMA) scaffolds were prepared and used as the negative control in this study. Scaffolds were implanted subcutaneously on the dorsum of male CD31 mice for 7, 21 and 30 days and the levels of tissue invasion, host tissue reaction and vascularization were evaluated histologically.

Sample Preparation: Washed poly(MAA-BMA) and poly(BMA) scaffolds were cut into disks 6 mm in diameter and 2 mm thick using a biopsy punch and razor blade. Endotoxin was removed (as described in Example 1) from the scaffolds and tested to be <0.25 EU/mL. Prior to implantation, the scaffolds were hydrated in sterile saline overnight (0.9% NaCl).

Implantation Procedure: Subcutaneous pockets were created in the right and left dorsal upper quandrants of male CD31 mice by blunt dissection. A poly(MAA-BMA) disk was then placed in the left quadrant pocket while a poly(BMA) control disk was placed in the right quadrant pocket for each mouse (FIG. 11). Surgical staples were removed 10 days after surgery upon complete closure of the incision wound. For each study time, 4 animals were implanted with both a poly(MAA-BMA) test and poly(BMA) control scaffold disk. At 7, 21 and 30 days post-implantation, the mice were sacrificed and the scaffold disks were explanted and fixed in 10% neutral buffered formalin for 24-48 h prior to tissue processing.

Histology and Immunohistochemistry Preparation: Specimens were prepared, cut and stained for hematoxylin and eosin (H+E) and vonWillebrand factor (factor VIII) by the clinical research pathology lab at Toronto General Hospital. Implants were removed from the formalin solution, embedded in paraffin and sectioned by cutting along the longitudinal axis at several points along the thickness of the disk. Samples from these sections were cut to a thickness of 4 μm prior to histological or immunohistochemical staining.

For H+E staining, sections were first dewaxed in 4 changes of xylene, then rehydrated with sequential dips in decreasing graded alcohol, followed by a water wash for 1 min. The sections were then placed in filtered hematoxylin for 5 min followed by a 2 min water wash. The sections were then decolorized in 1% acid alcohol and washed with water for 15 sec. Next, the samples were dipped 3 times in ammonia water, followed by a water wash for 1 min, placement in eosin for 10-15 sec and another quick rinse in water. The samples were dehydrated by sequential dips in increasing graded alcohol. Finally, the sections were dipped into 4 changes of xylene and mounted in Permount®.

For anti-vonWillebrand factor staining, the initial steps of dewaxing in xylene and rehydrating in sequential dips of decreasing graded alcohol were the same as described above. Then endogenous peroxidase activity was blocked with 3% aqueous hydrogen peroxide for 15 min, followed by a tap water wash. Pre-treatment was achieved with 1% pepsin for 15 min, followed by treatment with 10% normal goat serum. Next, the sections were incubated with an anti-vonWillebrand primary antibody (also referred to as factor VIII, rabbit anti-human polyclonal) at a dilution of 1/8000 for 1 h. The sections were then incubated with the secondary linking antibody, a goat-anti-rabbit antibody, for 30 min. Sections were then incubated for 30 min in Signet USA Level 2 labeling reagent, diluted ¼ with DAKO antibody diluting buffer. The sections were developed with NovaRed for 5 min and a counterstain with Mayer's hematoxylin was added. Dehydration was performed via increasing graded alcohol dips, followed by clearance with xylene and mounting in Permount®.

Microvessel Counting Method: The level of vascularization in the tissue invading the porous poly(MAA-BMA) and poly(BMA) scaffold explants was quantified using a microvessel density (MVD) count technique adapted from the tumour research literature. At low power (50×magnification), the three areas of the sample with the most abundant staining (“hotspots”) per section were identified with the scaffold. At high power (200×magnification), the number of factor VIII stained structures was counted for each “hot spot”. Any brown-staining endothelial cell or cluster of cells was counted as an individual microvessel if it was clearly separated from adjacent microvessels by other non-staining cells or connective tissue. The presence of a patent lumen or erythrocytes was not a requirement for the definition of a microvessel. MVD counts were expressed as microvessels per mm² with a mean MVD count per section calculated by averaging the three counts. The mean MVD counts were used to make a statistical comparison between the poly(MAA-BMA) test and poly (BMA) control scaffolds.

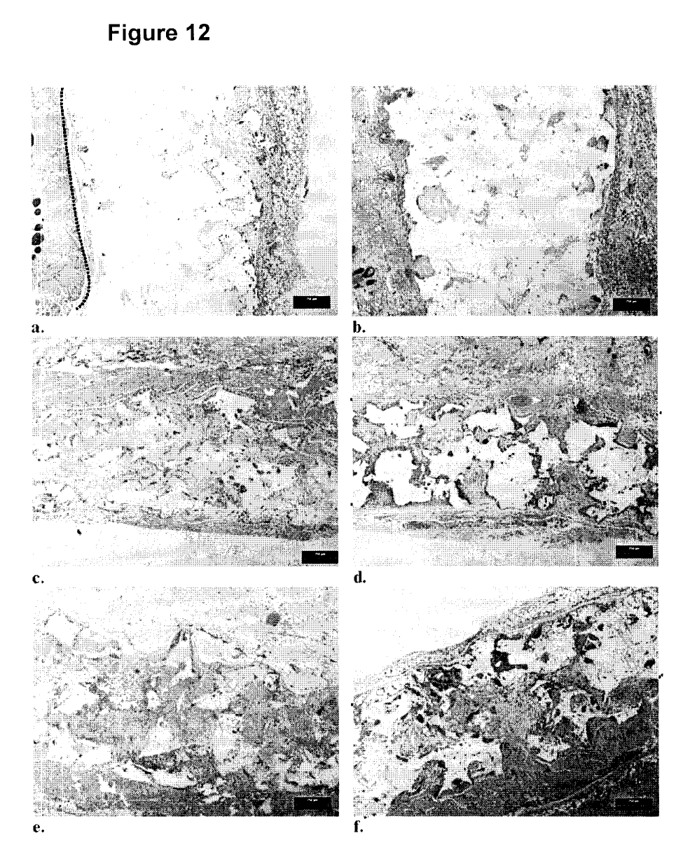

Characterization of Tissue Invasion into Scaffolds: Both the poly(MAA-BMA) test and poly(BMA) control scaffolds elicited a similar progression of tissue invasion over 30 days, as seen in FIG. 12. At 7 days ((a) and (b)), tissue penetration at the periphery of the scaffold was observed with minimal progression into the inner pores of the scaffolds. At 21 days ((c) and (d)) post-implantation, tissue had penetrated from the periphery to deeper sections of the scaffold. By 30 days((e) and (f), complete tissue infiltration throughout the scaffolds was apparent. Tissue penetrating from opposite sides of the scaffold merged to create a continuous bridge across the cross-section of the scaffold. However, even at 30 days there were regions of all scaffolds that appeared to be devoid of tissue indicating the presence of a fraction of closed pores in the scaffolds.

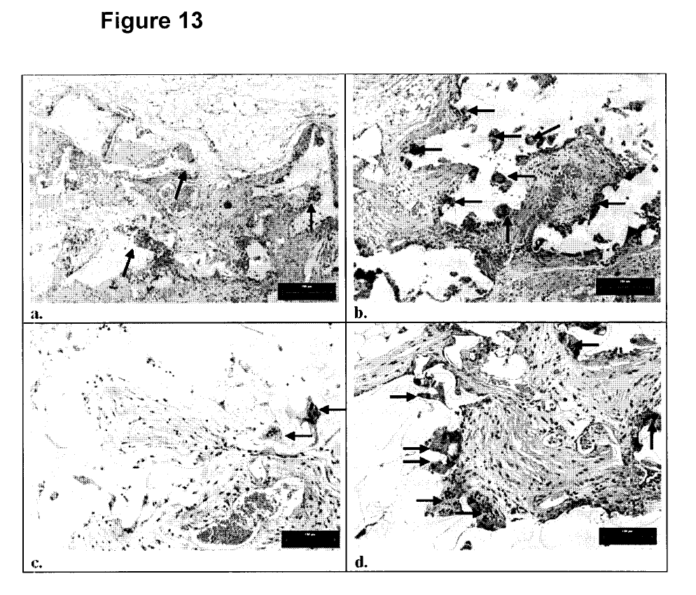

The inflammatory/foreign body response to the implanted scaffolds was also evaluated histologically. In all animals, after 7 days both test and control scaffolds were surrounded by a thin capsule containing proliferating fibroblasts, collagen fibers, capillary sprouts and some inflammatory cells. From this capsule, endothelial cells, fibroblasts and inflammatory cells penetrated into the porous cavities at the periphery of the scaffold. Very few giant cells (multinucleated macrophages) were observed at the border of the scaffold. There was however, a difference in the invading tissue of the test and control scaffolds at 21 and 30 days post-implantation. In the poly(MAA-BMA) scaffold explants, the invading tissue consisted mainly of fibroblasts, collagen and newly formed capillaries with some macrophages and a few giant cells. In contrast, the poly(BMA) control scaffold presented a more inflammatory response (FIG. 13). Along with fibroblasts, collagen and newly formed capillaries in the invading tissue, a larger number of neutrophils and foreign body giant cells were observed.

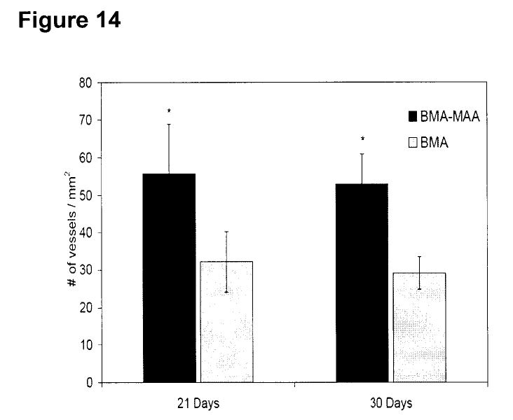

Characterization of Scaffold-Induced Vascularization: The microvessel density counting technique was used to quantify the level of histological vascularization in tissue penetrating the pores of the poly(MAA-BMA) and poly(BMA) scaffold explants. MVD counts in the tissue penetrating the pores of the poly(MAA-BMA) scaffolds at 21 and 30 days post-implantation were significantly higher than in the poly(BMA) scaffold (FIG. 14). There was no significant difference in MVD counts at 21 and 30 days.

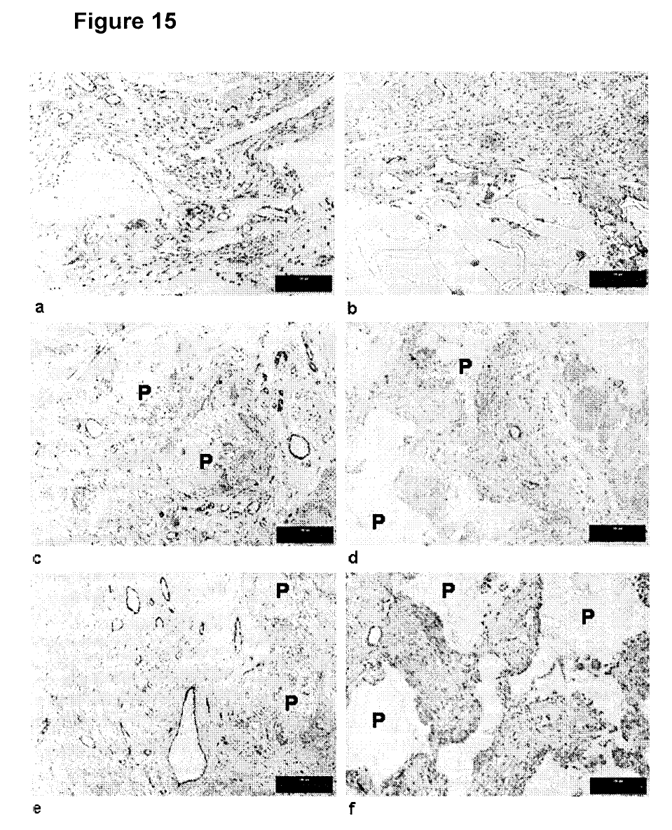

Photomicrographs of fVIII-stained sections of poly(MAA-BMA) scaffold explants show an increased level of brown-staining blood vessels compared with the poly(BMA) control scaffolds at all time points investigated (FIG. 15). MVD counts were not performed on sections at 7 days post-implantation as there was limited tissue ingrowth at this time. However, a large number of stained blood vessels can be seen at the periphery of the poly(MAA-BMA) scaffold at day 7, suggesting angiogenic activity soon after implantation.

In this study a poly(MAA-BMA) tissue engineering scaffold was fabricated and evaluated for its ability to enhance vascularization in the invading host tissue. Scaffolds implanted subcutaneously in mice revealed a higher number of fVIII stained blood vessels in tissue with close proximity to the copolymer. Microvessel density counts revealed a higher number of vessels in the tissue invading the pores of the poly(MAA-BMA) scaffolds compared to a poly(BMA) control. These results suggest that poly(MAA-BMA) is a pro-angiogenic biomaterial that may serve as a tissue engineering scaffold.

The above-described embodiments of the present invention are intended to be examples only. Alterations, modifications and variations may be affected to the particular embodiments by those of skill in the art without departing from the scope of the invention, which is defined solely by the claims appended hereto.

1. A pro-angiogenic porous polymer scaffold, said polymer comprising at least 20 mol-% monomeric subunits containing acidic functional groups, said polymer having a porosity of at least 40%, and having interconnected pores.

2. The scaffold of claim 1, wherein the acidic functional groups are selected from the group consisting of: carboxylic acids, carboxylates, sulfonic acids, sulfonates, phosphoric acids, and phosphates.

3. The scaffold of claim 1, wherein polymerizable monomeric subunits containing acidic functional groups used to produce the pro-angiogenic polymer are selected from the group consisting of methacrylic acid, acrylic acid, monoacryloxyethyl phosphate, 2-propene-1-sulfonic acid, 4-vinyl benzoic acid, crotonic acid, itaconic acid, vinylsulfonic acid, vinyl acetic acid, citric acid, styrene sulfonic acid, and sodium styrene sulfonate.

4. The scaffold of claim 3, wherein polymerizable monomeric subunits containing acidic functional groups used to produce the pro-angiogenic polymer are methacrylic acid.

5. The scaffold of claim 1, wherein the polymer is a polyacrylate.

6. The scaffold of claim 1, wherein the polymer is crosslinked.

7. The scaffold of claim 6, wherein the crosslinks are biostable.

8. The scaffold of claim 6, wherein the crosslinks are biodegradable.

9. The scaffold of claim 1, wherein the polymer is a graft polymer comprising a backbone and arms grafted onto the backbone, wherein the arms contain the at least 20 mol-% monomeric subunits containing acidic functional groups.

10. A method for making a pro-angiogenic porous polymer scaffold, wherein said polymer comprises at least 20 mol-% monomeric subunits containing acidic functional groups, said scaffold having a porosity of at least 40%, and having interconnected pores, said method comprising:
mixing one or more types of monomers, and an initiator together in a solvent, wherein at least 20 mol-% of said monomers contain an acidic functional group;
pouring the mixture over a fused salt bed having a pore size range of 10 to 800 microns;
allowing the mixture to polymerize; and
leaching the salt out, to yield the porous scaffold.

11. The method of claim 10, wherein the polymer is crosslinked, and the mixing step includes mixing in a crosslinking agent.

12. A method for tissue regeneration, comprising applying the scaffold of claim 1, to the vascularized tissue to be regenerated.

13. The method of claim 12, wherein the scaffold is pre-seeded with cells.
